(19) United States
(12) Patent Application Publication
(10) Pub. No.: US 2015/0353446 A1
(43) Pub. Date: Dec. 10, 2015

(54) PASSIVATION OF A ZEOLITE CATALYST IN A FLUIDIZED BED

(71) Applicant: BASF SE, Ludwigshafen (DE)

(72) Inventors: Mathias FEYEN, Hirschberg (DE); Bernd SCHAACK, Bensheim (DE); Ulrich MUELLER, Neustadt (DE); Thomas HEIDEMANN, Viernheim (DE); Veronika WILL, Graben-Neudorf (DE); Rainer SENK, Ludwigshafen (DE)

(73) Assignee: BASF SE, Ludwigshafen (DE)

(21) Appl. No.: 14/759,522

(22) PCT Filed: Feb. 26, 2014
(86) PCT No.: PCT/EP2014/053702

§ 371 Date: Jul. 7, 2015

(30) Foreign Application Priority Data
Mar. 27, 2013 (EP) 13161388.7

(51) Int. Cl. C07C 2/76; B01J 33/00; B01J 29/46

(52) U.S. Cl. CPC C07C 2/76; B01J 29/46; B01J 33/00; C07C 2529/46

(57) ABSTRACT

Process for producing a passivated catalyst comprising a zeolite and at least one active metal, wherein
the catalyst is held in a fluidized bed and
is passivated in the fluidized bed by means of a silicon compound.

The present invention relates to a process for producing a passivated catalyst comprising a zeolite and at least one active metal, wherein
the catalyst is held in a fluidized bed and
is passivated in the fluidized bed by means of a silicon compound.

Benzene can be prepared from methane or other low alkanes by dehydroaromatization, as described, for example, in WO 2009/124960. For this purpose, the alkanes are reacted over a catalyst at high temperatures, e.g. from 300 to 1000° C. The catalyst can be present, for example, as a fixed bed or fluidized bed in the reactor.

At the high temperatures, the catalyst naturally has to meet particular requirements. It has to remain active for as long as possible even at these high temperatures and ensure a very high conversion at a very high selectivity. At these high temperatures, formation of carbon in the form of carbonaceous material which deposits on the catalyst easily occurs. In particular, acidic sites on the catalyst surface promote the formation of carbon and deposition thereof.

According to U.S. Pat. No. 6,552,243, catalysts whose surface has been passivated by means of a silicon compound to form a silicon layer are used for the dehydroaromatization. The passivation of zeolite catalysts by means of silicon compounds is also known from WO 2007/080240 and WO 2005/014169. In the passivation process described in WO 2005/014169, the active metals of the catalyst are introduced after passivation; here, passivation can be carried out using liquid or gaseous passivating agents. In the process described in WO 2007/080240, the catalyst is treated in a fixed bed with a stream of nitrogen to which tetraethoxysilane has been added as passivating agent. WO 2007/080240 does not describe any dehydroaromatizations.

It is basically desirable to reduce formation of carbonaceous material and deposits on catalysts in dehydroaromatization further and achieve a further increase in the life of the catalyst with high yield and selectivity. At the same time, the process used for passivation should be simplified.

It was therefore an object of the present invention to provide a simplified process for passivation of catalysts and a simplified and improved process for dehydroaromatization using a passivated catalyst. In particular, formation of carbonaceous material and deposits during dehydroaromatization should be avoided.

We have accordingly found the process defined at the outset. We have also found a process for dehydroaromatization in which a catalyst which has been passivated in this way is used.

The catalyst comprises a zeolite.

Zeolites are naturally occurring or synthetically produced microporous substances having a three-dimensional framework structure composed of neutral SiO₄ tetrahedra and negatively charged AlO₄ tetrahedra and optionally further metal-oxygen compounds in the form of tetrahedra. Preference is given to zeolites which comprise more than 80% by weight, particularly preferably more than 95% by weight, neutral SiO₄ tetrahedra and negatively charged AlO₄ tetrahedra.

According to the different composition of the unit cell, a distinction is made between various basic types which are denoted by 3 letters according to an internationally recognized nomenclature of the International Zeolite Association, e.g. MFI, EUO, MTT.

Preference is given to a zeolite of the structure type MFI.

Zeolites of the structure type MFI are 10-ring zeolites, i.e. the circumference of the pores corresponds to a ring made up of a total of 10 atoms of Si, Al and optionally another metal; these 10 atoms are bridged by oxygen.

Known zeolites of the structure type MFI are, for example, TS-1 or ZSM-5. Particular preference is given to ZSM-5.

The pore diameter of the ZSM-5 is in general uniformly about 5.5 Angstrom.

ZSM-5 comprises predominantly SiO₄ tetrahedra and comprises only small amounts of negatively charged aluminum tetrahedra. The Si/Al ratio preferably corresponds to a ratio of SiO₂ to Al₂O₃ of from 10:1 to 200:1.

Cations balancing the negatively charged aluminum tetrahedra are generally hydrogen (acid H form) or alkali metal cations or ammonium cations.

Preference is given here to a zeolite in the H form. The zeolite in the H form can also comprise small amounts of alkali metal cations; the content of alkali metal cations is preferably less than 1% by weight, in particularly less than 0.1% by weight and particularly preferably less than 0.01% by weight.

Synthetic zeolites can be prepared from an aqueous solution comprising an Si compound (Si source, e.g. any silica such as pyrogenic or precipitated silica, water glass, silica gel, silanes or siloxanes), an aluminum compound (AI source, e.g. aluminum hydroxide), a template and optionally further additives, in particular to set the pH. It is possible in principle to introduce active metals during the preparation of the zeolite from the Si source and Al source.

The template is an organic compound which serves as space reserver for the pores. The solution is heated, forming a solid from the constituents. The organic compound (template) is finally removed by calcination (heating to very high temperatures), and only now are the pores present in the solid freely accessible. Suitable templates are, for example, allyltripropylammonium hydroxide or tetrapropylammonium hydroxide.

The catalyst preferably comprises a binder in addition to the zeolite.

Possible binders are Si-comprising binders, e.g. colloidal silicon dioxide, polysiloxanes or mixtures thereof.

In a preferred embodiment, the zeolite is firstly mixed with the binder. In particular, zeolite and binder are mixed in the form of liquid preparations (solutions or dispersions). The proportion of binder can be, for example, from 5 to 200 parts by weight per 100 parts by weight of zeolite, in particular from 10 to 100 parts by weight per 100 parts by weight of zeolite.

After mixing the zeolite with the binder, a shaping step can be carried out; here, the mixture is processed by the methods known to those skilled in the art to give shaped bodies. Shaping processes which may be mentioned are, for example, spraying of a suspension comprising the zeolite or the catalyst composition to give powders, tableting, pressing in the moist or dry state and extrusion. Two or more of these processes can also be combined. Auxiliaries such as pore formers and pasting agents or other additives known to those skilled in the art can be used for shaping. Possible pasting agents are compounds which lead to an improvement in the mixing, kneading and flow properties. For the purposes of the present invention, these are preferably organic, in particular hydrophilic polymers such as cellulose, cellulose derivatives such as methylcellulose, starch such as potato starch, wallpaper paste, acrylates, polyacrylates, polymethacrylates, polyvinyl alcohols, polyvinylpyrrolidone, polyisobutylene, polytetrahydrofuran, polyglycol ethers, fatty acid compounds, wax emulsions, water or mixtures of two or more of these compounds. For the purposes of the present invention, pore formers which may be mentioned are, for example, compounds which can be dispersed, suspended or emulsified in water or aqueous solvent mixtures, e.g. polyalkylene oxides, polystyrene, polyacrylates, polymethacrylates, polyolefins, polyamides, polyesters, carbohydrates, cellulose, cellulose derivatives such as methylcellulose, natural sugar fibers, pulp, graphite or mixtures of two or more of these compounds. Pore formers and/or pasting agents are preferably removed from the shaped body obtained after shaping by means of at least one suitable drying and/or calcination step.

The shaped bodies obtained can be powders having a desired distribution of the powder size or shaped bodies having a uniform, defined geometry. Thus, the catalysts can be, for example, spherical (hollow or solid), cylindrical (hollow or solid), ring-, saddle-, star-, honeycomb- or pellet-shaped. Furthermore, extrudates having, for example, a rod, trilobe, quatrolobe, star or hollow-cylindrical shape are possible. Furthermore, the catalyst composition to be shaped can be extruded, calcined and the extrudates obtained in this way can be crushed and processed to give crushed material or powder. The crushed material can be separated into various sieve fractions. A preferred sieve fraction has a particle size of from 0.25 to 0.5 mm.

In a preferred embodiment, a powder is produced from the mixture of binder and zeolite, in particular by spray drying.

The catalyst comprises at least one catalytically active metal. The catalyst preferably comprises a plurality of catalytically active metals.

The term metals as used here refers to metals in elemental form or else in the form of metal ions or central atoms of complexes. Particular preference is given to metal ions which are present as salts.

The catalytically active metals can be any metals of the Periodic Table.

In a preferred embodiment, the catalyst comprises one or more active metals selected from among Mo, Mn, Cr, Zr, V, Zn, Cu, Ni, Fe, W, Ga, Ge and Co.

The catalyst particularly preferably comprises one or more active metals selected from among Mo, Ni, Cu, Fe, Zn.

The catalyst very particularly preferably comprises Mo and additionally a further metal or a plurality of further metals. In a very particularly preferred embodiment, the catalyst comprises Mo and additionally a further metal or a plurality of further metals selected from among Fe, Cu, Ni, Zn.

The catalyst produced after all process steps a) to c) preferably comprises from 1 to 20% by weight, particularly preferably from 3 to 20% by weight, of active metals, based on the total weight of the catalyst.

In a preferred embodiment, the catalyst produced after all process steps comprises from 1 to 15% by weight of molybdenum (Mo), in particular from 3 to 12% by weight of Mo, and from 0 to 10% by weight, preferably from 0.5 to 5% by weight, of other active metals, e.g. those mentioned above, preferably Fe, Cu, Ni, Zn.

The active metals can be introduced into the catalyst before or after passivation by means of the silicon compound, e.g. they can be applied beforehand to the zeolite or to the mixture of zeolite and binder or the shaped body produced therefrom, in particular powder. They can also be applied only after passivation of the catalyst by means of the silicon compound. This subsequent application of the active metals is customary in order to prevent the active metals from also being passivated.

In a particular embodiment, more than 80% by weight, in particular more than 90% by weight, particularly preferably more than 98% by weight and in particular 100% by weight, of the total active metals comprised in the catalyst produced by the process of the invention are introduced into or applied to the catalyst before passivation.

The processes described below for applying the metals apply in the same way regardless of whether the active metals are applied to the zeolite, the mixture of zeolite and binder or the shaped body produced therefrom, in particular powder, or before or after passivation.

The active metals can be applied wet-chemically or dry-chemically.

Wet-chemically, the active metal can be applied in the form of aqueous, organic or organic-aqueous solutions of its salts or complexes by impregnating the zeolite or catalyst with the respective solution. Supercritical CO₂ can also serve as solvent. The impregnation can be carried out by the incipient wetness method in which the porous volume of the zeolite is filled with an approximately equal volume of impregnation solution and, optionally after aging, the support is dried. It is also possible to employ an excess of solution, in which case the volume of this solution is greater than the porous volume of the zeolite. Here, the zeolite is mixed with the impregnation solution and stirred for a sufficiently long time. It is also possible to spray the zeolite with a solution of the salt of the active metal. Other production methods known to those skilled in the art, for example precipitation of the active metal on the zeolite, spraying-on of a solution comprising a compound of the active metal, sol impregnation etc., are also possible. In the case of molybdenum, particularly suitable compounds are (NH₄)₆Mo₇O₂₄, ammonium heptamolybdate (NH₄)₂Mo₂O₇, MoO₂, MoO₃, H₂MoO₄, Na₂MoO₄, Mo-oxalates with Mo in various oxidation states, (NH₃)₃Mo(CO)₃ and Mo(CO)₆. After application of the active metal, the catalyst is dried at from about 80 to 130° C., usually for from 4 to 20 hours under reduced pressure or in air.

The elements Mn, Cr, Zr, V, Zn, Cu, Ni, Fe, W, Ga, Ge and Co are preferably applied wet-chemically. As metal salts, preference is given to using the nitrates such as copper nitrate, nickel nitrate, iron nitrate and cobalt nitrate, but other salts known to those skilled in the art for wet-chemical application can also be used. These include the halides, in particular chloride, acetate, alkaline carbonates, formate, tartrate, acetate, complexes with ligands such as acetylacetonate, amino alcohols, EDTA, carboxylates such as oxalate and citrate and also hydroxycarboxylic acid salts.

In an embodiment, the solution by means of which the active metal or active metals is/are applied comprises at least one complexing agent. The complexing agent is preferably selected from the group consisting of acetylacetonate, amino alcohols, EDTA, carboxylates such as oxalate and citrate and also hydroxycarboxylic acid salts. Particular preference is given to using EDTA.

Dry-chemically, the active metal can, for example, be applied at relatively high temperatures from the gas phase by deposition onto the zeolite or the catalyst. In the case of molybdenum, gaseous Mo(CO)₆, for example, is suitable for this purpose.

The catalyst is held in a fluidized bed and passivated in the fluidized bed by means of a silicon compound. Here, a silicon compound is a compound which comprises at least one silicon atom.

The catalytic reaction occurs at the acid sites in the pores of the catalyst. The acid sites outside the pores, i.e. on the freely accessible surface of the catalyst, promote undesirable formation of carbonaceous material on the surface of the catalyst. These acid sites can be passivated by reaction with a silicon compound and formation of a polymeric silicon layer, in general a silicon dioxide layer.

Possible silicon compounds are, in particular, those which can be converted by polycondensation or polyaddition, e.g. at elevated temperature, into polymeric silicon compounds, in particular those having a silicon dioxide base structure.

Preference is given to nonpolymeric silicon compounds having a molecular weight of less than 5000 g/mol, in particular less than 1000 g/mol and particularly preferably less than 500 g/mol.

The silicon compounds preferably have a molecule diameter which is at least larger than the diameter of the pores of the zeolite used. In the case of the preferred zeolite ZSM-5, the silicon-comprising compounds therefore have a diameter of at least greater than 5.5 Angstrom.

Suitable silicon compounds which may be mentioned are, in particular, silanes, siloxanes or silazanes.

Silanes are silane (SiH₄) and its derivatives, i.e. compounds in which at least one hydrogen has been replaced by another substituent. Preference is given to silanes in which from one to four H atoms have been replaced by organic groups, halogens or hydroxy groups. Possible organic groups are, for example, alkyl groups, aryl groups, alkoxy groups or aroxy groups. Preference is given to at least two of the organic groups being groups which condense with elimination of water to form a polymeric compound having an Si—O—Si base structure. Particular preference is given to silanes having from 2 to 4 alkoxy groups, preferably C1-C10-alkoxy groups or C1-C4-alkoxy groups. Mention may be made in particular of tetraalkoxysilanes such as tetramethoxysilane or tetraethoxysilane.

Siloxanes are compounds having two Si atoms connected via an oxygen atom. The two Si atoms are substituted by H atoms or organic groups. As regards the organic groups, what has been said above with regard to the silanes applies analogously. The siloxanes preferably comprise at least two organic groups which undergo a condensation reaction; in particular, they are alkoxy groups as mentioned above.

Silazanes are compounds having two Si atoms which are connected via a nitrogen group and have the base structure

(R−)₃Si—NH—Si(—R)₃

The radicals R are preferably organic groups, e.g. alkyl groups or alkoxy groups. Suitable silazanes are, for example, hexaalkylsilazanes, e.g. hexa-C1-C10-alkylsilazanes. Mention may be made by way of example of hexamethylsilazane:

(CH₃—)₃Si—NH—Si(—CH₃)₃.

The catalyst is held in a fluidized bed during passivation.

For this purpose, the catalyst is generally firstly introduced as fixed bed into a reactor and gas, referred to here as carrier gas, is passed into it from below.

The flow of the carrier gas is increased until the fluidized bed is formed. The gas flow is set so that a stable fluidized bed is formed, i.e., in particular, that the fluidized bed is not carried out of the reactor but remains at the desired height.

The silicon compound can then be applied, preferably in gaseous form, to the catalyst. For this purpose, the silicon compound can be heated to temperatures above its boiling point and brought into contact with the catalyst in the fluidized bed.

Preference is given to bringing a mixture of the silicon compound with other gases, preferably the carrier gas, into contact with the catalyst in the fluidized bed.

The gas, which is preferably at the same time the carrier gas, can be an inert gas such as nitrogen or noble gases or the gaseous starting materials of the later reaction, e.g. methane or natural gas.

In a preferred embodiment, the gas is an inert gas, particularly preferably nitrogen or helium.

In a particularly preferred embodiment, the gas can be brought into contact with the silicon compound beforehand so as to take up the silicon compound, preferably to saturation of the carrier gas stream with the silicon compound, and subsequently brought into contact with the fluidized bed of the catalyst. The silicon compound does not have to be heated to its boiling point for this purpose. In general, the vapor pressure at room temperature is sufficient for the gas or carrier gas to take up enough silicon compound.

The gas or carrier gas preferably comprises from 0.01 to 10 percent by volume, in particular from 0.1 to 2 percent by volume, of the silicon compound.

The dilution of the silicon compound with another gas, preferably the carrier gas, makes it possible to ensure good distribution of the silicon compound on the surface.

The catalyst which has been treated in this way with the silicon compound in the fluidized bed can subsequently be removed from the reactor and optionally dried. Drying can, for example, be carried out in a separate process step at temperatures of from 20 to 150° C. and optionally under reduced pressure, e.g. under vacuum, before the further reaction of the silicon compound to form a polymeric silicon layer.

The reaction of the silicon compound to form a polymeric silicon layer is preferably carried out at elevated temperature.

The reaction to form the polymeric silicon layer can, for example, be carried out at temperatures of from 100 to 800° C., in particular from 200 to 700° C., particularly preferably from 300 to 700° C. (calcination).

The temperature is, in this case, usually increased slowly over a relatively long period of time and the maximum temperature reached is maintained over a relatively long period of time. The total period of time can be, for example, from 2 to 20 hours.

The surface-passivated catalyst which is finally obtained preferably has a content of from 0.001 to 5% by weight, particularly preferably from 0.01 to 1% by weight, of Si of the silicon compound or the reaction product obtained therefrom after a final calcination. The amount indicated above is based only on the Si atom of the silicon compound since the content of the silicon introduced by the silicon compound does not change even on further reaction of this silicon compound.

Use of the catalyst

The catalyst obtained by the above production process is preferably used as catalyst for dehydroaromatization. In particular, the catalyst is used for the dehydroaromatization of alkanes and alkenes.

The dehydroaromatization is preferably the dehydroaromatization of a feed stream comprising C1-C4-aliphatics to form benzene and possibly higher aromatics. The C1-C4-aliphatics can be, for example, methane, ethane, propane, n-butane, i-butane, ethene, propene, 1- and 2-butene or isobutene.

The dehydroaromatization is in particular a process for preparing benzene from methane or mixtures of aliphatics which comprise more than 70% by weight, particularly preferably more than 90% by weight, based on the total amount of aliphatics, of methane. In particular, natural gas can be used as methane or mixture of aliphatics.

Gaseous compounds which do not dehydroaromatize, e.g. hydrogen, water, carbon monoxide, carbon dioxide, nitrogen or noble gases, can additionally be mixed into the feed stream. Inert gases such as nitrogen or noble gases are used in order to reduce the partial pressure. Other gases such as carbon monoxide or carbon dioxide may decrease formation of carbonaceous material.

Preference is given to a dehydroaromatization under nonoxidative conditions. For this purpose, the concentration of oxidants such as oxygen or nitrogen oxides in the feed stream should preferably be below 5% by weight, more preferably below 1% by weight, particularly preferably below 0.1% by weight. The mixture is very particularly preferably free of oxygen and nitrogen oxides.

The catalyst can optionally be activated beforehand. Activation is generally carried out at temperatures lower than those in the later reaction and with a defined temperature/time profile in order to rule out chemical reactions in or on the catalyst as completely as possible. The activity of the catalyst may be able to be increased by such an activation.

To activate the catalyst, the catalyst is preferably brought into contact with a gas having an appropriate temperature. A prior activation can, for example, be carried out by means of a C1-C4-alkane such as methane, ethane, propane, butane or a mixture thereof, preferably methane. The activation can be carried out at a temperature of from 250 to 650° C., preferably from 350 to 550° C., and a pressure of from 0.5 to 100 bar, preferably from 1 to 50 bar, in particular from 1 to 10 bar. The GHSV (gas hourly space velocity) in the activation is usually from 100 to 4000 h⁻¹, preferably from 500 to 2000 h⁻¹.

The catalyst can also be activated by means of an Hz-comprising gas stream; the H₂ gas stream can additionally comprise inert gases such as N₂, He, Ne and Ar.

Preference is given to carrying out an activation using a C1-C4-alkane, optionally in admixture with hydrogen. The activation is particularly preferably carried out using methane, optionally in admixture with hydrogen.

The dehydroaromatization of C1-C4-aliphatics can be carried out in the presence of the above-described catalysts at temperatures of from 400 to 1000° C., preferably from 500 to 900° C., particularly preferably from 600 to 800° C., in particular from 650 to 800° C., at a pressure of from 0.5 to 100 bar, preferably from 1 to 50 bar, particularly preferably from 1 to 30 bar, in particular from 1 to 10 bar. The feed stream can be introduced into the reactor at, for example, a GHSV (gas hourly space velocity) of from 100 to 10 000 h⁻¹, preferably from 200 to 3000 h⁻¹.

If the activity of the catalysts decreases, they can be regenerated by conventional methods known to those skilled in the art. A possible method is, in particular, regeneration of the catalysts by means of hydrogen.

For this purpose, the reaction can be stopped and the catalyst can be regenerated by means of hydrogen. Reaction cycle and regeneration cycle can alternate and the feed stream and hydrogen can accordingly be passed alternately over the catalyst.

Hydrogen can advantageously be added to the feed stream so that regeneration occurs simultaneously with the reaction. In particular, the feed stream in the regeneration phase can comprise more than 10% by volume, in particular more than 30% by volume and particularly preferably more than 50% by volume, of hydrogen.

Reactors suitable for carrying out the dehydroaromatization are, for example, tube reactors or shell-and-tube reactors. The catalyst produced according to the invention can be present as a fixed bed or fluidized bed in these reactors.

The catalyst produced by the process of the invention can be used for carrying out the dehydroaromatization of C1-C4-aliphatics, in particular methane, with high yields and selectivities. In particular, high yields of and selectivities to benzene are achieved. The deposition of carbonaceous material on the catalyst is significantly reduced by the passivation method according to the invention; as a result, the life is increased and the time intervals between necessary regeneration of the catalyst are considerably lengthened.

### Examples

A catalyst composed of zeolite H-ZSM-5 and a polysiloxane as binder was used for the examples. The catalyst was present as powder having a particle size of 45 μm-200 μm. After drying and calcination, the catalyst comprised 78% by weight of H-ZSM-5, with the balance being SiO₂ formed from the binder.

The catalyst produced in this way was used in the example and comparative example described below.

### Comparative Example

### Passivation of the Catalyst by Impregnation and Subsequent Introduction of Active Metals

Passivation by Impregnation:

150 g of the catalyst were added to a solution of 1.73 g of hexamethylsilazane in 180 ml of tetrahydrofuran. The entire solution was taken up by the catalyst.

The catalyst which had been treated in this way was dried at 35° C. under reduced pressure in a drying oven for 4 hours and subsequently calcined (3 hours at 500° C. and 4 hours at 500° C.).

Loading with Active Metals:

Solution 1:

15.44 g of ammonium heptamolybdate tetrahydrate were placed in a glass beaker and dissolved in a total of 135.2 ml of deionized water.

Solution 2:

6.93 g of nickel(II) nitrate hexahydrate were placed in a glass beaker and dissolved in a total of

135.2 ml of deionized water.

130 g of the passivated catalyst were impregnated with solution 1 until the entire solution had been taken up by the catalyst; the impregnated catalyst was dried at 120° C. for 16 hours.

The passivated catalyst was subsequently additionally impregnated with solution 2 until the entire solution had been taken up; the impregnated catalyst was again dried at 120° C. for 16 hours and subsequently calcined (heated to 500° C. over a period of 3 hours and after reaching the 500° C. held for 4 hours).

### Example

### Passivation in the Fluidized Bed and Subsequent Introduction of Active Metals

200 g of the catalyst were introduced into a fluidized-bed reactor. Hexamethyldisilazane was introduced into a separate apparatus, with the temperature of the hexamethylsilazane being room temperature (about 20° C.). 30 standard liters (standard I)/h of nitrogen were passed over the hexamethylsilazane so as to give a gas mixture composed of nitrogen and hexamethylsilazane.

Introducing this gas mixture into the fluidized-bed reactor formed the fluidized bed of the catalyst. The gas mixture at the same time served as carrier gas for the fluidized bed. The temperature in the fluidized bed was about 100° C. The gas flow of 30 standard liters (standard I)/h was maintained for one hour.

The catalyst which had been treated in this way was dried at 120° C. for 16 hours and subsequently calcined

(heated to 500° C. over a period of 3 hours and after reaching the 500° C. held for 4 hours).

Loading with Active Metals

Solution 1:

22.32 g of ammonium heptamolybdate tetrahydrate were placed in a glass beaker and dissolved in a total of 199.28 ml of deionized water.

Solution 2:

10.03 g of nickel(II) nitrate hexahydrate were placed in a glass beaker and dissolved in a total of

199.28 ml of deionized water.

188 g of the passivated catalyst were impregnated with solution 1 until the entire solution had been taken up by the catalyst; the impregnated catalyst was dried at 120° C. for 16 hours.

The passivated catalyst was subsequently additionally impregnated with solution 2 until the entire solution had been taken up; the impregnated catalyst was again dried at 120° C. for 16 hours and subsequently calcined

(heated to 500° C. over a period of 3 hours and after reaching the 500° C. held for 4 hours).

Nonoxidative Dehydroaromatization of Methane

The experiments were carried out using 100 g of the catalyst from the example and alternatively from the comparative example in a fluidized-bed reactor.

A methane stream was firstly passed at a flow rate of 100 standard liters (standard I)/h through the reactor and the temperature was slowly increased to the reaction temperature (700° C.) during this (this activated the catalyst; the Mo oxide comprised is carbidized to Mo carbide). The flow rate was calculated for atmospheric pressure and standard temperature.

The reaction was subsequently carried out using a mixture of CH₄/He (90:10) at a flow of 20 standard I/h. The temperature in the reactor was 700° C. and the pressure was 2.5 bar. One reaction cycle took 10 hours. After each reaction cycle, the catalysts were regenerated by introduction of hydrogen at 4 bar and 750° C. for a time of 5 hours (regeneration cycle).

Each trial comprised about 10 reaction cycles and 10 regeneration cycles.

In each reaction cycle, a constant reaction was achieved after a start-up time of a few hours and the following values were determined by taking samples:
the total conversion of methane into carbon compounds in % by weight
the proportion of benzene in the carbon compounds formed, in % by weight
the proportion of carbonaceous material in the carbon compounds formed, in % by weight

The values were in each case identical in all reaction cycles of a trial and are summarized in the table below:

| | Trial using | Trial using |
|---|---|---|
| | catalyst from | catalyst from |
| | the example | the comparative example |
| % by weight of | 7.5 | 7.5 |
| reacted methane | | |
| Proportion of | 85% | 80 |
| benzene | | |
| Proportion of | <5 | 10 |
| carbonaceous material | | |

1. A process for producing a passivated catalyst, the process comprising:
holding a catalyst in a fluidized bed and
passivating the catalyst in the fluidized bed by a silicon compound to produce the passivated catalyst,
wherein the passivated catalyst comprises a zeolite and an active metal.

2. The process according to claim 1, wherein the zeolite is a zeolite of a MFI structure.

3. The process according to claim 1, wherein the zeolite is ZSM-5.

4. The process according to claim 1, wherein the passivated catalyst further comprises a binder.

5. The process according to claim 1, wherein the passivated catalyst comprises at least one active metal selected from the group consisting of Mo, Mn, Cr, Zr, V, Zn, Cu, Ni, Fe, W, Ga, Ge and Co.

6. The process according to claim 1, wherein
the passivated catalyst obtained comprises from 1 to 20% by weight of the active metal and
more than 80% by weight of the active metal has been introduced into the catalyst before passivation.

7. The process according to claim 1, wherein the silicon compound is a silazane, silane or siloxane.

8. The process according to claim 1, wherein the silicon compound is a hexaalkylsilazane.

9. The process according to claim 1, wherein the catalyst is brought into contact with a gas mixture comprising the silicon compound and a further gas in the fluidized bed.

10. The process according to claim 1, further comprising:
contacting a gas with the silicon compound, thereby obtaining a gas comprising the silicon compound and
subsequently contacting the gas comprising the silicon compound with the fluidized bed of the catalyst.

11. The process according to claim 10, wherein the gas is a carrier gas of the fluidized bed.

12. The process according to claim 1, wherein the passivated catalyst comprises from 0.001 to 5% by weight of Si of the silicon compound or a reaction product obtained therefrom after a final calcination.

13. A process for dehydroaromatizing an alkane, a alkene, or a mixture thereof, the process comprising:
dehydroaromatizing the alkane, the alkene, or the mixture thereof in the presence of the passivated catalyst obtained from the process according to claim 1.

14. A process for preparing benzene, the process comprising:
preparing the benzene from methane or a mixture comprising more than 70% by weight of methane in the presence of the passivated catalyst obtained from the process according to claim 1.
